(19) United States
(12) Patent Application Publication
(10) Pub. No.: US 2005/0075371 A1
(43) Pub. Date: Apr. 7, 2005

(54) Methods and compositions for the oral administration of prodrugs of proton pump inhibitors

(75) Inventors: Jie Shen, Irvine, CA (US); Devin F. Welty, Foothill Ranch, CA (US); Diane D. Tang-Liu, Newport Beach, CA (US)

Correspondence Address: Brent A. Johnson;Allergan, Inc., 2525 Dupont Drive, Irvine, CA, 92612, US

(73) Assignee: ALLERGAN, INC., Irvine, CA (US)

(21) Appl. No.: 10/943,411

(22) Filed: Sep. 17, 2004

Related U.S. Application Data
(60) Provisional application No. 60/508,356, filed on Oct. 3, 2003.
(60) Provisional application No. 60/513,880, filed on Oct. 22, 2003.

(51) Int. Cl. A61K 31/4439

(52) U.S. Cl. 514/338

(57) ABSTRACT

Oral dosage forms, methods of treating diseases or adverse conditions, and methods of inhibiting gastric acid secretion related to prodrugs of a proton pump inhibitor are disclosed herein. Certain embodiments relate to the membrane permeability of the proton pump inhibitor and/or the membrane permeability of the prodrug. Other embodiments relate to prodrugs comprising an acidic functional group and a sulfonyl moiety. In other embodiments, the prodrug is a carboxylic acid which comprises a phenylsulfonyl moiety. Other embodiments relate to the pH of dosage forms and dosage forms comprising salts of acidic functional groups.

### CROSS-REFERENCE TO RELATED APPLICATIONS

This application claims priority to U.S. Provisional Patent Application No. 60/508,356, filed Oct. 3, 2003. This application also claims priority to U.S. Provisional Patent Application No. 60/513,880, filed Oct. 22, 2003. Both priority documents are expressly incorporated by reference herein.

### BACKGROUND OF THE INVENTION

1. Field of the Invention

The present invention is directed to oral dosage forms and methods comprising prodrugs of proton pump inhibitors, which are useful as inhibitors of gastric acid secretion.

2. Description of the Related Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042 and 5,708,017. Generally speaking, the benzimidazole-type inhibitors of gastric acid secretion are believed to work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), ESOMEPRAZOLE (U.S. Pat. No. 6,369,085) PANTOPRAZOLE (U.S. Pat. No. 4,758,579), and RABEPRAZOLE (U.S. Pat. No. 5,045,552). Some of the diseases treated by proton pump inhibitors and specifically by the five above-mentioned drugs include peptic ulcer, heartburn, reflux esophagitis, erosive esophagitis, non-ulcer dyspepsia, infection by Helicobacter pylori, alrynitis and asthma.

Whereas the proton pump inhibitor type drugs represent a substantial advance in the field of human and veterinary medicine, they are not totally without shortcomings or disadvantages. For example, it is believed that the short systemic half-life of the drug limits the degree of gastric acid suppression currently achieved. Furthermore, it appears that the short plasma half-life of the drug may contribute to significant gastric pH fluctuations that occur several times a day in patients undergoing PPI therapy. Additionally, PPIs are acid-labile, and in most cases it is necessary to enterically coat the drug in order to prevent the acidic milieu of the stomach from destroying the drug before the drug is absorbed into systemic circulation. Thus, any contribution that might improve the acid stability or plasma half-life of the presently used proton pump inhibitors will be a significant improvement in the art.

As further pertinent background to the present invention, applicants note the concept of prodrugs which is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buuret al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomsen et al.).

A publication by Sih., et al. (Journal of Medicinal Chemistry, 1991, vol. 34, pp 1049-1062), describes N-acyloxyalkyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N—H group. This publication provides no data nor suggestion regarding the duration of the inhibitory activity of these prodrugs.

U.S. Pat. No. 6,093,734 and PCT Publication WO 00/09498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylori effects. PCT Publication WO 02/00166 describes compounds that are said to be nitric oxide (NO) releasing derivatives of proton pump inhibitors of the benzimidazole structure.

Copending U.S. patent application Ser. No. 10/620,252, filed Jul. 15, 2003 discloses prodrugs of the proton pump inhibitor type drugs having an arylsulfonyl group with an acidic functional group attached, which provided improved solubility in physiological fluids and improved cell penetration.

### BRIEF DESCRIPTION OF THE INVENTION

We have surprisingly discovered that the oral administration of certain prodrugs of proton pump inhibitors can prolong the systemic half-life of the proton pump inhibitor. While not intending to be bound in any way by theory, it is believed that oral administration of the prodrug results in increased systemic half-life of the proton pump inhibitor because the prodrugs of the present invention are absorbed more slowly from the gastrointestinal tract into the bloodstream than the proton pump inhibitors.

We have also discovered certain methods that can be used to stabilize these prodrugs in solid and liquid dosage forms.

Some embodiments relate to oral dosage forms comprising a prodrug of a proton pump inhibitor. In certain embodiments, the membrane permeability of the proton pump inhibitor is more than twice the membrane permeability of the prodrug. In these embodiments, the dosage form has a pH from 3 to 9.

In other embodiments related to oral dosage forms, the prodrug comprises an acidic functional group and a sulfonyl moiety. In these embodiments, at least 10% of the acidic functional group is in the form of a pharmaceutically acceptable salt.

Other embodiments relate to methods of inhibiting gastric acid secretion in a person. These embodiments comprise orally administering a prodrug of a proton pump inhibitor to the person, wherein the prodrug has a membrane permeability which is less than 5×10⁻⁷ cm/sec.

Other embodiments relate to methods of treating a disease or adverse condition affecting the gastrointestinal tract in a person. These embodiments comprise administering orally to the person a prodrug of a proton pump inhibitor wherein the prodrug is a carboxylic acid which comprises a phenylsulfonyl moiety. In these embodiments, the carboxylic acid is in a dosage form comprising at least 1% of said carboxylic acid in the form of a pharmaceutically acceptable salt.

### BRIEF DESCRIPTION OF THE FIGURES

FIG. 1 is a plot of the systemic half-life (T_{1/2}) of proton pump inhibitors omeprazole and lansoprazole, following oral administration of their corresponding prodrugs in dog, as a function of membrane permeability of the prodrugs, measured as the permeability coefficient (Papp) across Caco-2 cells in the apical to basolateral direction.

### DETAILED DESCRIPTION OF THE INVENTION

The term “oral dosage form” used in relation to this invention should be interpreted to mean any form of solid or liquid which is intended to be administered orally to a person.

The term “prodrug” has the meaning previously described herein, and in relation to this disclosure refers to a prodrug of a proton pump inhibitor. The term “proton pump inhibitor” also has the meaning previously described herein.

The term “membrane permeability” used in relation to this disclosure refers to the value obtained by carrying out the procedure described in Example 1 herein. While not intending to limit the scope of the invention in any way, it is believed that the membrane permeability obtained by the procedure of Example 1 is a good relative quantitative measurement of the ability of a given compound to diffuse through a membrane in a living system such as the gastrointestinal lining of a human. While a direct correlation between the two properties may not necessarily be made, the relative trend in membrane permeability among compounds in a series will be consistent with the relative trend in the ability of the compounds in a series to pass through the gastrointestinal lining.

As stated previously, in one embodiment the membrane permeability of the proton pump inhibitor is more than twice the membrane permeability of the prodrug. In another embodiment, the membrane permeability of the proton pump inhibitor is more than 10 times the membrane permeability of the prodrug. In another embodiment the membrane permeability of the proton pump inhibitor is more than 100 times the membrane permeability of the prodrug. In another embodiment the membrane permeability of the proton pump inhibitor is more than 150 times the membrane permeability of the prodrug.

In another embodiment the membrane permeability of the prodrug is less than 1×10⁻⁶ cm/sec. In another embodiment the membrane permeability of the prodrug is less than 5×10⁻⁷ cm/sec. In another embodiment the membrane permeability of the prodrug is less than 1×10⁻⁷ cm/sec. In another embodiment the membrane permeability of the prodrug is less than 5×10⁻⁸ cm/sec.

In certain embodiments, pH is an important consideration in formulating oral dosage forms. While not intending to be bound in any way by theory, we have surprisingly discovered that certain pH ranges have additional advantages in terms of stability and solubility of the prodrugs. We have found that prodrugs of the present invention are hygroscopic, in that they gain water over time when stored in a dry solid form. Thus, even when the prodrugs are administered in a solid dosage form, pH stability of the compounds is often important because the absorbed water could be involved in acid and base catalyzed hydrolysis, or related reactions, which could decompose the prodrug and adversely affect the shelf-life of the dosage form. As such, it is important to point out that many prodrugs disclosed herein have improved stability in dosage forms having a pH of from 3 to 9 relative to the stability of these prodrugs in dosage forms having a pH which is outside of this range. In certain cases, the stability of some of the prodrugs disclosed herein may be further improved when the pH is between 5 and 8.

The term “pH of an oral dosage form” should be interpreted broadly in relation to the claims presented herein. In the case of a liquid dosage form, the term pH has the meaning broadly understood in the art, that is, the pH is the negative log of the hydrogen or hydronium ion concentration. However, the property of pH is also meaningful in relation to solid dosage forms for the purposes of this disclosure. In the case of a solid dosage form, the pH of the dosage form is defined as the result obtained by the following test.
1. The dosage form is ground to a fine powder.
2. The dosage form is added to an equal weight of water, and the mixture is mixed vigorously enough that all soluble material has substantial contact with the water.
3. The mixture is filtered, or the liquid is decanted out.
4. The pH of the solution is measured.

In certain embodiments disclosed herein, the pH of the solid dosage form comprising such therapeutically active agents is from 3 to 9. In other embodiments, the dosage form has a pH from 5 to 8. In other embodiments, the dosage form has a pH from 6 to 8.

Many of the embodiments disclosed herein relate to prodrugs comprising an acidic group. An “acidic functional group” as used herein refers to an oxygen containing functional group which has a pKₐ below 10. Thus, while not intending to limit the scope of the claims in any way an acidic functional group may include an organic acid such as a carboxylic acid, a phosphonic acid, or a sulfonic acid.

Acidic functional groups can be in one of two forms, the acid form or the salt form, depending upon whether the particular group has undergone an acid-base reaction. The two forms of these functional groups may also be known by other names. The acid form may also be known as the protonated form, nonionized form, or the neutral form. The salt form may also be known as the deprotonated form, the ionized form, the anionic form, or the conjugate base form.

While not intending to limit the scope of the invention in any way, these acidic functional groups may be important in facilitating formulation by improving the solubility of the prodrug. While not intending to limit the scope of the invention in any way, or to be bound in any way by theory, these acidic functional groups also have an additional benefit in that they help improve the stability of the prodrug by helping to buffer the formulation to the more stable pH range. While not intending to limit the scope of the invention in any way, the carboxylic acid is a particularly useful acidic functional group in this regard. The term “carboxylic acid” has the broadest meaning normally understood by practitioners of the chemical arts. While not intending to be bound or limited in any way by theory, it is believed that if a part of the prodrug in the formulation is in the form of the pharmaceutically acceptable salt of a carboxylic acid, the prodrug can help to keep the pH high enough to improve the stability of the formulation. For example, if at least 1% of the carboxylic acid is in the form of a pharmaceutically acceptable salt, the pH of the formulation will not be lower than the pKa of the acid by more than two pH units. If at least 10% of the carboxylic acid is in the form of a pharmaceutically acceptable salt, the pH of the formulation will not be lower than the pKa of the acid by more than one pH unit. If 50% of the acid is in the form of the pharmaceutically acceptable salt, the pH of the formulation will be equal to the pKa of the acid. Finally, if at least 90% of the carboxylic acid is in the form of a pharmaceutically acceptable salt, the pH of the formulation will be at least one pH unit higher than the pKa of the acid.

A “pharmaceutically acceptable salt” is any salt that retains the activity of the parent compound and does not impart any deleterious or untoward effect on the subject to which it is administered and in the context in which it is administered.

Pharmaceutically acceptable salts of acidic functional groups may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

Methods of preparation of dosage forms having known amounts of salts is well known in the art. For example, while not intending to be limiting, a person may take a given quantity of a carboxylic acid, and add an amount of a base equal to 0.1 molar equivalents of the acid to give a mixture where 10% of the carboxylic acid is in the form of a pharmaceutically acceptable salt. In addition, methods of determining the quantity of the salt form of an acidic functional group are well known in the art. Such methods include, but are not limited to titration and spectroscopic methods.

In certain embodiments disclosed herein, the prodrug is not enterically coated. The term “enterically coated” means the prodrug or the dosage form comprising the prodrug is coated by a coating which protects the prodrug from the acids present in the stomach, but which coating disintegrates in the higher pH environment of the intestines. In many dosage forms, small particles of the prodrug are coated with the enteric coating. In other dosage forms, an entire capsule, tablet, or other solid dosage form is coated with the enteric coating. While not intending to be bound in any way by theory, it is believed that the prodrugs disclosed herein are sufficiently stable in the presence of the acidic milieu of the stomach that enteric coating of the prodrug is generally not necessary. This is believed to be a significant contribution to the art because enteric coatings are typically expensive, and, while not intending to be bound in any way by theory, because enteric coatings limit the drug absorption by not allowing it to be absorbed in the stomach.

Certain compounds have been shown to be useful as prodrugs in relation to the embodiments disclosed herein. In certain embodiments, the prodrug comprises a sulfonyl moiety. A “sulfonyl” moiety is defined herein as a moiety comprising an SO₂ group, where a sulfur atom is directly covalently bonded to two oxygen atoms. In other embodiments, the prodrug comprises a phenylsulfonyl moiety. The term “phenylsulfonyl” moiety should be broadly interpreted to mean any moiety where the sulfur of the SO₂ group is directly covalently bonded to a carbon that is part of a phenyl ring. The term “phenyl ring” should be broadly understood to mean any ring comprising six carbon atoms having three conjugated double bonds. Thus, a phenylsulfonyl moiety could be monosubstituted, meaning that the sulfonyl group is the only group directly attached to the phenyl ring, or the phenylsulfonyl moiety could have from 1 to 5 additional substituents which are not a hydrogen atom, and are directly attached to a carbon of the phenyl ring. In certain embodiments, the prodrug comprises both a phenylsulfonyl moiety and a carboxylic acid or a pharmaceutically acceptable salt thereof.

While not intending to limit the scope of the invention in any way, in many situations one practicing the invention might choose a prodrug which would be converted after administration into one of the widely used and well tested commercially available proton pump inhibitors (PPI) such as lansoprazole, esomeprazole, omeprazole, pantoprazole, and rabeprazole. In situations where one of the commercially available PPIs is used as the PPI in practicing this invention, one practicing the invention may want to consider circumstances related to the individual to which the prodrug is administered in making decisions related to the practice of the invention. For example, if the person to which the prodrug is being administered is known to respond well to omeprazole, then one may consider using a prodrug of omeprazole in relation to the practice of the invention. In another situation, a person may have a history of being effectively treated by lansoprazole, in which case one may consider using a prodrug of lansoprazole in practicing the invention. The specific aspects of the invention related to proton pump inhibitor are given merely to provide guidance and direction to one practicing the invention, and are not intended to limit the overall scope of the invention in any way. In one embodiment the proton pump inhibitor is lansoprazole. In another embodiment the proton pump inhibitor is omeprazole. In another embodiment the proton pump inhibitor is pantoprazole. In another embodiment the proton pump inhibitor is rabeprazole.

Certain embodiments relate to particular structures, which are useful as prodrugs.
One embodiment comprises 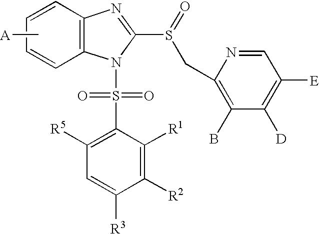
or a pharmaceutically acceptable salt thereof
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

In another embodiment related to the one just described, R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

In certain embodiments, the prodrug has a structure comprising

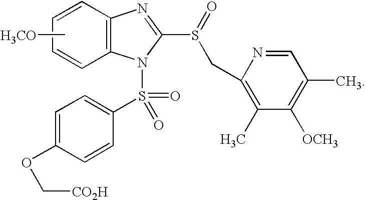

In other embodiments, the prodrug has a structure comprising

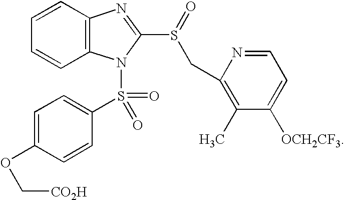

In other embodiments, the prodrug has a structure comprising

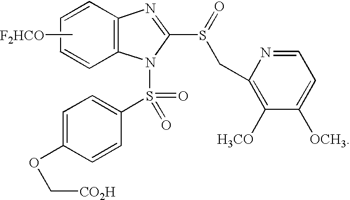

In other embodiments, the prodrug has a structure comprising

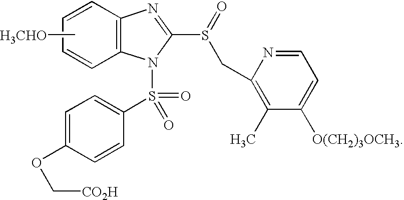

The prodrugs of the present invention can be prepared by the methods described in the following U.S. Patent documents, all of which are expressly incorporated by reference herein: U.S. Pat. No. 6,093,734; U.S. patent application Ser. No. 09/783,807, filed Feb. 14, 2001; the U.S. Pat. App. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. Pat. App. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number. However, these methods are only given to provide guidance, and are not meant to limit the scope of the invention in any way. One of ordinary skill in the art will recognize that there are many ways in which the prodrugs of the present invention can be prepared without departing from the spirit and scope of the present invention.

Those skilled in the art will readily understand that for oral administration the compounds of the invention are admixed with pharmaceutically acceptable excipients which per se are well known in the art. Specifically, a drug to be administered systemically, it may be confected as a powder, pill, tablet or the like, or as a syrup or elixir suitable for oral administration. Description of the substances normally used to prepare tablets, powders, pills, syrups and elixirs can be found in several books and treatise well known in the art, for example in Remington's Pharmaceutical Science, Edition 17, Mack Publishing Company, Easton, Pa.

Prodrugs of the present invention can be combined with certain amounts of the proton pump inhibitors to which they are related to provide a drug-prodrug combination, and the combination administered for inhibition of gastric acid secretion. Thus, certain embodiments relate to a mixture of the prodrug and the proton pump inhibitor. Other embodiments relate to the administration of both the prodrug and the proton pump inhibitor. While not intending to limit the scope of these embodiments, it is believed that the proton pump inhibitor (drug) initially inhibits gastric acid secretion of the patient, and as the effective concentration of the proton pump inhibitor (drug) is decreased by metabolism, the prodrug is used to maintain a sustained presence of a therapeutically effective systemic concentration of the proton pump inhibitor. In certain embodiments the ratio of the molar concentration of the prodrug to the molar concentration of the proton pump inhibitor is from 1 to 1000.

In certain embodiments related to the combined use of the proton pump inhibitor and the prodrug, the membrane permeability of the proton pump inhibitor is more than twice the membrane permeability of the prodrug. In other embodiments, the membrane permeability of the proton pump inhibitor is more than 10 times the membrane permeability of the prodrug. In other embodiments, the membrane permeability of the proton pump inhibitor is more than 100 times the membrane permeability of the prodrug. In other situations, the membrane permeability of the proton pump inhibitor is more than 150 times the membrane permeability of the prodrug.

In other situations, two prodrugs of a proton pump inhibitor are administered to a person. Other embodiments comprise a mixture of two different prodrugs of a proton pump inhibitor. In some situations, it is advantageous to have one prodrug which has a high membrane permeability relative to the second prodrug. Thus, similar to the drug-prodrug case cited earlier, both fast action and sustained release can be achieved. In one embodiment, the two prodrugs have a membrane permeability ratio which is 2 or more. In another embodiment, the two prodrugs have a membrane permeability ratio which is from 2 to 10. In another embodiment, the two prodrugs have a membrane permeability ratio which is 10 or more. In another embodiment, the two prodrugs have a membrane permeability ratio which is 10 to 100. In another embodiment, the two prodrugs have a membrane permeability ratio which is 100 or more. In another embodiment, the two prodrugs have a membrane permeability ratio which is from 100 to 500. The membrane permeability ratio in relation to these embodiments is defined as the value of the membrane permeability of the prodrug having the higher membrane permeability, divided by the membrane permeability of the prodrug having the lower membrane permeability. In certain embodiments the ratio of the molar concentration of the two prodrugs is from 1 to 1000.

The following examples provide guidance and direction in making and using the invention, and to demonstrate the advantages of the present invention. However, except in the case of Example 1, they are not to be interpreted as limiting the scope of the invention in any way. In the case of Example 1, it should only be interpreted as limiting in relation to those claims where membrane permeability is used as a limitation.

### Test Compounds

Membrane permeability and oral bioavailability tests were carried out for the compounds shown in Table 1 below. The generic structure, I, is shown as a combination of a proton pump inhibitor (X) and a sulfonyl-bearing moiety which is attached to the proton pump inhibitor to form the prodrug according to the formula below. The identity of each group represented by R¹-R⁵ is shown in the table.

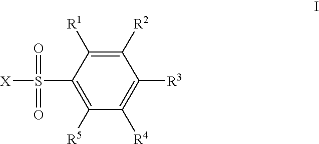

The different possibilities for X are shown below.

TABLE 1

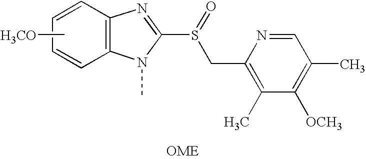

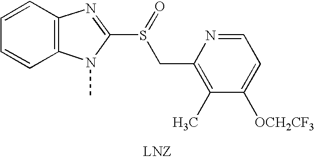

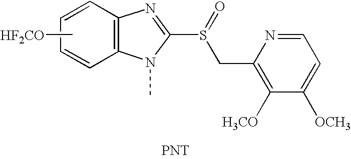

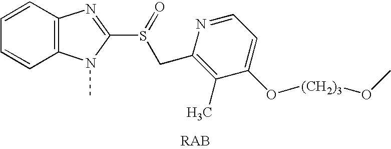

| Compound | X | R¹ | R² | R³ | R⁴ | R⁵ |
|---|---|---|---|---|---|---|
| 1 | OME | H | H | OCH₂CO₂H | H | H |
| 2 | OME | CH₃ | H | OCH₂CO₂H | H | CH₃ |
| 3 | OME | H | H | OCH₂C(CH₃)₂CO₂H | H | H |
| 4 | OME | CH₃ | H | OCH₂C(CH₃)₂CO₂H | H | CH₃ |
| 5 | OME | H | H | CH₂CO₂H | H | H |
| 6 | OME | H | CO₂H | H | H | H |
| 7 | LNZ | H | CO₂H | H | H | H |
| 8 | LNZ | H | CO₂H | OCH₃ | H | H |
| 9 | LNZ | H | H | CH₂CO₂H | H | H |
| 10 | LNZ | H | H | OCH₂CO₂H | H | H |
| 11 | LNZ | H | H | OCH₂C(CH₃)₂CO₂H | H | H |
| 12 | LNZ | H | CH₂CO₂H | CH₂CO₂H | H | H |
| 13 | LNZ | H | CO₂H | H | H | CH₃ |
| 14 | LNZ | H | CO₂H | H | H | OCH₃ |
| 15 | LNZ | CH(CH₃)₂ | H | CH₂CO₂H | H | H |
| 16 | LNZ | H | OCH₂CO₂H | CO₂H | H | H |
| 17 | LNZ | CH(CH₃)₂ | H | OCH₂CO₂H | H | CH₃ |
| 18 | LNZ | H | H | CO₂H | H | H |
| 19 | LNZ | H | (CH₂)₂CO₂H | CH₃ | H | H |
| 20 | OME | H | H | OCH₂CO₂CH₃ | H | H |
| 21 | OME | H | H | OCH₂CO₂NH₂ | H | H |
| 22 | OME | H | CO₂H | CO₂H | H | H |
| 23 | OME | H | CO₂H | OCH₂CO₂H | H | H |
| 24 | OME | H | OCH₂CO₂H | OCH₂CO₂H | H | H |
| 25 | OME | OCH₃ | H | CO₂H | H | H |
| 26 | OME | H | | CO₂H | H | H |
| 27 | OME | H | CO₂H | H | H | CH₃ |
| 28 | PNT | H | H | OCH₂CO₂H | H | H |
| 29 | PNT | H | CO₂H | H | H | CH₃ |
| 30 | RAB | H | CO₂H | H | H | H |
| 31 | RAB | H | CO₂H | H | H | CH₃ |
| 32 | RAB | CH₃ | H | OCH₂CO₂H | H | CH₃ |
| 33 | RAB | H | H | CO₂H | H | H |
| 34 | LNZ | CH₃ | H | OCH₂CO₂H | H | CH₃ |
| 35 | LNZ | H | OCH₂CO₂H | OCH₂CO₂H | H | H |
| 36 | LNZ | H | H | CO₂H | H | H |
| 37 | LNZ | CH₃ | H | CO₂H | H | H |
| 38 | LNZ | H | (CH₂)₂CO₂H | OCH₃ | H | H |
| 39 | OME | CH₃ | H | OCH₂CONH₂(CH₂)₅ | H | CH₃ |
| | | | | CO₂CH₃ | | |
| 40 | OME | H | H | OCH₂CONH₂(CH₂)₅ | H | H |
| | | | | CO₂CH₃ | | |
| 41 | OME | H | H | (CH₂)₂CO₂H | H | H |
| 42 | OME | H | (CH₂)₂CO₂H | OCH₃ | H | H |

Compounds were prepared according to procedures described the U.S. patent application Ser. No. 10/620,252, filed Jul. 15, 2003 and U.S. patent application Ser. No. 10/487,340, filed Jul. 15, 2003 incorporated by reference herein.

Omeprazole and lansoprazole were purchased from Sigma (St. Louis, Mo.).

### EXAMPLE 1

Determination of membrane permeability in all examples described herein was accomplished by the following procedure. This procedure is also used to determine whether a given prodrug falls within the scope of those claims given herein which relate to membrane permeability.

Materials/Methods

| | |
|---|---|
| Test System: | Cultured Caco-2 cells |
| Seeding Density: | 2 × 10⁵ cells/cm² in Costar 12 well Transwell ™ |
| | plates |
| Culture Age: | 17-21 days post seeding |
| Source: | American Type Culture Collection, Manassas, |
| | VA |
| Growth Media: | Dulbecco's Modified Eagle Media (DMEM) |
| | (Gibco BRL) supplemented with 10% fetal bovine |
| | serum and 0.1% nonessential amino acids |
| Dosing Formulation: | 10 μM proton pump inhibitor or prodrug in |
| | DMEM. Make on the day of dosing. |
| Assay: | LC-MS/MS |

Bi-Directional Transport Experiment:

Caco-2 cells were seeded on Costar™ 12 mm diameter, 0.4 μm pore size transwell filters, and were cultured at 37° C., 5% CO₂ in a humidified tissue culture chamber.

DMEM was equilibrated as a transport buffer in 37° C. water bath an hour before experiment. The cells were then equilibrated in transport buffer for 1 hr at 37° C.

Dosing solution (10 μM) was prepared by adding a 20 μL aliquot of a 10 mM stock solution of the prodrug to 20 mL of transport buffer.

Test Conditions:

Transport across Caco-2 cell monolayer was measured at 37° C., in the apical to basolateral direction (n=3).

Transport buffer was removed from both apical and basolateral compartment of filters. Dosing solution (0.2 mL) was added to the apical compartment of the cell layers on transwell filters, and 0.8 ml fresh pre-warmed transport buffer was added to basolateral compartment. Timing was started for transport, and at 5, 20, and 60 min after transport started, sample fluid (400 μL) was collected from the basolateral compartment. Fresh transport buffer (400 μL) was added back to the basolateral compartment, and the fluid was thoroughly mixed.

Transport samples, dosing solution, and standards(100 μL) each were mixed with 100 μl of a 500 ng/ml internal standard (Lansoprazole-D) for LC-MS/MS analysis, and part of each sample (100 μL) was vortexed and transferred into glass LC-MS/MS vials for analysis.

Data Analysis

The apparent permeability coefficient (Papp, cm/sec), otherwise known herein as the membrane permeability, is determined from the following relationship:
Papp=J/(ACₒ)
where J (pmol/min) is the transport rate, meaning the rate of prodrug movement through the cell layer, A (cm²) is the filter surface area, and Cₒ (μM) is the initial dosing concentration.

The transport rate J, is calculated as the slope of the linear regression fit for the transport amount over time data using Microsoft Excel® 97 SR-2 (Microsoft Corp. Redmond, Wash.).

Reference Standard:

Lucifer yellow (LY) was used as a paracellular permeability reference standard to determine integrity of cell layers used in the experiments. LY transport in the apical to basolateral direction was carried out in the same manner as described above. Fluorescence level in basolateral fluid sampled at 5, 20, and 60 min post dose was determined using Fluostar Galaxy (BMG Labtechnologies, Durham, N.C.) at excitation/emission wavelengths of 485/520 nm. A standard curve covering the range from 0.002 to 0.5 mg/mL is constructed to quantify the amount of LY in the transport sample to calculate permeability coefficient (Papp). Papp values below 1×10⁻⁶ cm/sec were considered acceptable and were used to normalize Papp values for test articles across experiments by multiplying the Papp values for the test articles by the factor x according to the following equation,
x=(1×10⁻⁶)/(S)
where S is the value of Papp obtained for LY.

### EXAMPLE 2

Oral bioavailability of omeprazole, lansoprazole, pantoprazole, rabeprazole, and test compounds was determined in rats (Sprague-Dawley) and dogs (beagle) by administering an oral solution to the animal and collecting serial blood samples through 24 hr post dose. Blood concentrations of the compounds omeprazole, lansoprazole, pantoprazole, rabeprazole, and test compounds were quantified using an achiral liquid chromatography tandem mass spectrometry method (LC-MS/MS). Systemic pharmacokinetic parameters were determined for omeprazole or lansoprazole using non-compartmental analysis in Watson® version 6.3, available from InnaPhase Corporation, Philadelphia, Pa. Results of the oral pharmacokinetic studies are presented in Tables 2A-2D below.

TABLE 2A
Systemic Omeprazole Half-life in Rats

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.31 |
| 1 | Oral | 10 | 1.7 |
| Omeprazole | Intravenous | 1 | 0.15 |
| 1 | Intravenous | 1 | 0.18 |

Table 2A shows the systemic half-life of omeprazole in rats after oral and intravenous administration of omeprazole and compound 1. Surprisingly, these results show that the systemic half-life of omeprazole after intravenous administration of omeprazole is nearly identical to that after intravenous administration of the prodrug (compound 1). The prodrug was not detected in the bloodstream 5 minutes after it was administered intravenously. These unexpected results demonstrate that in the case of compound 1, systemic conversion of the prodrug to omeprazole does not take an appreciable amount of time compared to the amount of time omeprazole is present systemically. By contrast, absorption of the prodrug from the gastrointestinal tract into the blood unexpectedly prolongs the systemic half-life of omeprazole to a significant extent relative to both the intravenous and oral administration of omeprazole. Table 2B shows a similar effect in dogs. Thus, these results show that oral administration of a prodrug will increase the systemic half-life of a proton pump inhibitor. While not intending to limit the scope of the invention, results that will be discussed later, and which are presented in Table 2D, indicate that a relationship may exist between the membrane perameability of the prodrug and the systemic half-life of the proton pump inhibitor.

TABLE 2B
Systemic Omeprazole Half-life in Dogs

| | | Equivalent | Systemic |
|---|---|---|---|
| Compound | Dosing | omeprazole | omeprazole |
| Administered | Route | dose (mg/kg) | half-life (hr) |
| Omeprazole | Oral | 10 | 0.70 |
| 1 | Oral | 10 | 2.4 |
| Omeprazole | Intravenous | 1 | 0.60 |
| 1 | Intravenous | 1 | 1.0 |

Table 2C summarizes the systemic half-lives of the prodrugs and the PPIs for compounds 1-42 in dogs and rats. While not intending to be limited or bound in any way by theory, these results demonstrate that slow absorption of the prodrug from the gastrointestinal tract can contribute to an increase in the systemic half-life of the proton pump inhibitor. For many of the prodrugs in the table, the systemic half-life of the prodrug (i.e. the intact prodrug molecule) is either very short relative to the systemic half-life of the proton pump inhibitor, or is so short that the intact prodrug cannot be detected in the blood, and thus the half-life cannot be detected (NC). By contrast, however, for many of these same prodrugs, the measured systemic half-life of the proton pump inhibitor is significantly increased relative to the orally administered prodrug. Since the hydrolysis of the prodrugs in the blood does not contribute significantly to the increased systemic half-life of the proton pump inhibitors, it follows that the absorption of the prodrug from the gastrointestinal tract is slowed sufficiently to prolong the systemic half-life of the proton pump inhibitor. Thus, while not intending to be bound or limited in any way by theory, in the case of these particular prodrugs, it is the absorption step rather than the hydrolysis step that is the rate-limiting step of the pharmacokinetic process. In other words, the gastrointestinal tract, rather than the bloodstream, acts as the depot for the prodrug. This is possible because the prodrugs disclosed herein are significantly more stable than the proton pump inhibitors in the acidic milieu of the stomach and in the neutral, aqueous, milieu of the intestines. This will be discussed further later herein.

TABLE 2C
Systemic Half-Life of Prodrugs and PPIs in Dogs and Rats

Dog

| | |
|---|---|
| T_{1/2} | Rat |

| | | PPI | | T_{1/2} |
|---|---|---|---|---|
| Compound | T_{1/2} | 0.696 | T_{1/2} | PPI |
| Omeprazole | Prodrug | (0.116) | Prodrug | 0.308 |
| 1 | NC | 2.08 | NC | 2.4 |
| | | (1.19) | | |
| 2 | 0.113 | 1.61 | | |
| | (n = 1) | | | |
| 3 | 0.311 | 0.813 | NC | 1.76 |
| | | | | (0.93) |
| 4 | 1.26 | 0.837 | 0.342 | 0.708 |
| | | | | (0.479) |
| 5 | 0.269 | 1.03 | NC | 1.7 |
| 6 | 0.303 | 1.91 | NC | 1.93 |
| | | | | (0.39) |
| 20 | NC | 2.70 | | |
| | | (0.62) | | |
| 21 | NC | 0.855 | 1.51 | 0.523 |
| | | (0.143) | (1.44) | (0.338) |
| 22 | NC | 3.89 | | |
| 23 | NC | 1.22 | NC | 2.72 |
| | | | | (1.35) |
| 24 | | 1.37 | NC | 0.384 |
| 25 | NC | 1.03 | | |
| 26 | 1.19 | 0.881 | | |
| 27 | 0.117 | 1.10 | NC | 2.17 |
| | (n = 1) | | | (0.53) |
| 39 | | | NC | 1.50 |
| | | | | (1.18) |
| 40 | | | NC | 2.69 |
| | | | | (0.76) |
| 41 | | | NC | 0.761 |
| | | | | (0.497) |
| 42 | | | 0.521 | 1.47 |
| | | | | (0.29) |
| Lansoprazole | | 0.573 | | 0.510 |
| | | (0.150) | | (0.168) |
| 7 | 0.206 | 0.893 | NC | 1.93 |
| | | | | (1.41) |
| 8 | NC | 1.08 | NC | 1.80 |
| | | | | (1.20) |
| 9 | NC | 0.894 | NC | 0.341 |
| | | | | (0.151) |
| 10 | NC | 0.989 | | |
| | | (0.307) | | |
| 11 | NC | 0.873 | NC | 0.933 |
| | | (0.288) | | (1.009) |
| 12 | NC | 0.931 | | |
| 13 | 0.122 | 1.77 | NC | 2.35 |
| | | | | (1.22) |
| 14 | 0.118 | 1.39 | | 0.536 |
| | | | | (0.217) |
| 15 | NC | 0.923 | | |
| 16 | NC | 1.00 | NC | 1.86 |
| | | | | (0.74) |
| 17 | 1.49 | 1.13 | | |
| 18 | 0.0899 | 0.909 | | |
| 19 | 1.84 | 0.484 | | |
| 34 | | | NC | 1.11 |
| | | | | (0.71) |
| 35 | | | NC | 1.84 |
| | | | | (0.87) |
| 36 | | | NC | 0.389 |
| | | | | (0.085) |
| 37 | | | NC | 2.19 |
| | | | | (0.80) |
| 38 | | | 1.04 (0.35) | 1.43 |
| | | | | (0.42) |
| Pantoprazole | | 0.743 | | 0.696 |
| | | | | (0.116) |
| 28 | NC | 2.61 | NC | 1.45 |
| | | | | (0.73) |
| 29 | NC | 0.958 | NC | 1.01 |
| | | | | (0.30) |
| Rabeprazole | | 0.369 | | |
| 30 | 1.12 | 0.491 | | |
| 31 | 0.843 | 0.855 | | |
| 32 | 0.526 | 1.52 | | |
| 33 | 0.746 | 0.894 | | |

Values in parenthesis indicate the standard deviation, when obtained.
NC: plasma concentration of prodrug was too low to calculate half-life, or undetected.

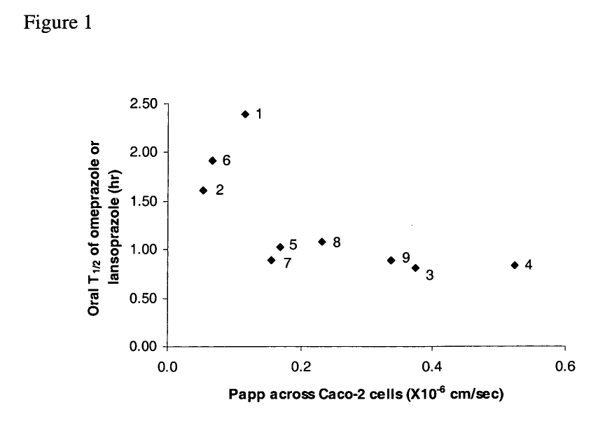

The results in Table 2D demonstrate the unexpected discovery that membrane permeability correlates with the systemic half-life of a PPI after oral administration of a PPI or a prodrug. They also demonstrate that membrane permeability is a good predictive test for how much a given prodrug will increase the systemic half-life of a PPI because the data shows that decreasing the membrane permeability of a prodrug increases the systemic half-life of the PPI. It should be noted that there is some scatter in the data, which is believed to be due to the relatively large random error in determining the systemic half-life. However, FIG. 1 is a plot that graphically demonstrates that despite the scatter, as a general trend, systemic half-life of a PPI resulting from oral administration of its prodrug increases with decreasing membrane permeability of the prodrug. It should be noted that the correlation is not expected to be linear, since membrane permeability is a rate term associated with the reciprocal of time, whereas half-life is a measurement of time. Thus, a reciprocal relationship between the two parameters might exist, meaning that one parameter might be a function of the reciprocal value of the other. While not intending to be bound in any way by theory, these results predict that if a prodrug has lower membrane permeability than a PPI, oral administration of the prodrug will result in a longer systemic half-life of the PPI relative to the systemic half-life resulting from oral administration of the PPI itself.

TABLE 2D
Membrane permeability of proton pump inhibitors
and their prodrugs, and their systemic half-life
in dogs after their oral administration.

| | | Permeability | |
|---|---|---|---|
| Compound | Parent PPI | (×10⁻⁶ cm/sec) | t_{1/2} (hours) |
| Omeprazole | — | 13 | 0.70 |
| 1 | Omeprazole | 0.12 | 2.4 |
| 2 | Omeprazole | 0.054 | 1.6 |
| 3 | Omeprazole | 0.38 | 0.81 |
| 4 | Omeprazole | 0.52 | 0.84 |
| 5 | Omeprazole | 0.17 | 1.0 |
| 6 | Omeprazole | 0.067 | 1.9 |
| Lansoprazole | — | 15 | 0.57 |
| 7 | Lansoprazole | 0.16 | 0.89 |
| 8 | Lansoprazole | 0.23 | 1.1 |
| 9 | Lansoprazole | 0.34 | 0.89 |

### EXAMPLE 3

The physicochemical properties of compound 1 were analyzed. Compound 1 was found to be hygroscopic, in that 9% weight gain was observed for the compound after 14 days of storage at 25° C. at 75% relative humidity.

TABLE 3A
Solubility Profile of Compound 1 at 25° C.
in Buffered Aqueous Solutions

| | | Solubility |
|---|---|---|
| pH | Buffer Composition | (mg/mL) |
| 1 | 0.1 M HCl | 1.8 |
| 3 | Citric Acid (0.1 M)/ | 0.4 |
| | Na₂HPO₄ (0.2 M) | |
| 5 | Citric Acid (0.1 M)/ | >50 |
| | Na₂HPO₄ (0.2 M) | |
| 7 | sodium phosphate (0.1- | >50 |
| | 0.2 M) | |
| 9 | sodium phosphate (0.1- | >50 |
| | 0.2 M) | |

The solubility profile of compound 1 in at various pH values is presented in Table 3A. This data shows that the aqueous solubility of the compound is significantly enhanced at around pH 5. While not intending to be bound in any way by theory, it is believed that this improvement in solubility is due to the deprotonation of a sufficient quantity of the acid. While not intending to be bound in any way by theory, this suggests that the prodrug should be significantly easier to formulate, particularly in the case of liquid dosage forms, when the pH is around 5 or higher.

TABLE 3A
Stability Profile of Compound 1 at 25° C.
in Buffered Aqueous Solutions

| | | Half-life | | Degradation |
|---|---|---|---|---|
| | Buffer | (t_{1/2}) | Shelf life | Rate Constant |
| pH | Composition | hours | (t_{90%}) hours | (k) 1/hours |
| 1 | 0.1 M HCl | 3.6 | 0.5 | 0.194 |
| 3 | Citric Acid (0.1 M)/ | 78.0 | 11.9 | 0.009 |
| | Na₂HPO₄ (0.2 M) | | | |
| 5 | Citric Acid (0.1 M)/ | 89.2 | 13.6 | 0.008 |
| | Na₂HPO₄ (0.2 M) | | | |
| 7 | sodium phosphate | 286.8 | 43.6 | 0.002 |
| | (0.1-0.2 M) | | | |
| 7.4 | sodium phosphate | 291.2 | 44.3 | 0.002 |
| | (0.1-0.2 M) | | | |
| 9 | sodium phosphate | 23.0 | 3.5 | 0.030 |
| | (0.1-0.2 M) | | | |
| 10 | sodium phosphate | 2.3 | 0.4 | 0.298 |
| | (0.1-0.2 M) | | | |

The aqueous stability data of compound 1 is presented in Table 3B. These results show that, the half-life (t_{1/2}), the shelf-life (t_{90%}), and the rate constant for degradation (k) for compound 1 are significantly improved in the pH range of 3-9. While not intending to be bound in any way by theory, these results suggest that formulation of dosage forms in the pH range of from 3 to 9 should greatly improve the stability of the prodrugs, thus improving shelf-life and facilitating formulation. Further, these results suggest that dosage forms having a pH from 6 to 8 will be particularly useful in certain situations.

Additionally, these results demonstrate that the prodrugs are significantly more stable in acidic and neutral aqueous solutions than the proton pump inhibitors. The stability of omeprazole and other proton pump inhibitors have been reported (Kromer et al., “Differences in pH-Dependent Activation Rates of Substituted Benzimidazoles and Biological in vitro Correlates”, Pharmacology 1998; 56:57-70; and Ekpe et al, “Effect of Various Salts on the Stability of Lansoprazole, Omeprazole, and Pantoprazole as Determined by High Performance Liquid Chromatograpy”, Drug Development and Industrial Pharmacy, 25(9), 1057-1065 (1999)), and while the stability is somewhat buffer dependent, typical half-lives for omeprazole are about 1 hour at pH 5 and about 40 hours at pH 7, which is about 1-2 orders of magnitude shorter than the prodrug half-lives presented in Table 3A. This instability of the proton pump inhibitors has generally necessitated their formulation in enterically-coated dosage forms. Thus, while not intending to limit the scope of the invention in any way, or to be bound in any way by theory, these results suggest that the prodrugs disclosed herein have sufficient stability to allow the gastrointestinal tract to act as a depot for the prodrug, and also have sufficient stability that the use of enteric coatings is not necessary for effective formulation of a dosage form.

### EXAMPLE 4

To further demonstrate that enteric-coating is unnecessary for the prodrugs disclosed herein, degradation of compound 1 in simulated gastric fluid at pH 1 was studied. Simulated gastric fluid was prepared as specified by USP (http://www.uspnf.com/uspnf/usp26nf21/default.htm, Reagents>Solutions>Test Solutions>Gastric Fluid, Simulated). To make 200 mL of simulated gastric fluid, 0.4 g of sodium chloride and 0.64 g of purified pepsin, with an activity of 800 to 2500 units per mg of protein, was dissolved in 1.4 mL of hydrochloric acid and sufficient water. The solution was adjusted to the appropriate pH with hydrochloric acid.

The pH dependence of the half-life of compound 1 in the simulated gastric fluid is depicted in Table 4A.

TABLE 4A
Half-life of Compound 1 in Simulated Gastric Fluid

| pH | Half-life (h) |
|---|---|
| 1.2 | 3 |

The bioavailability of compound 1 in enterically coated and non enterically coated dosage forms was investigated for dogs and monkeys. Regular and enteric-coated size 3 HPMC capsules (Capsugel, Morris Plains, N.J.) containing compound 1 were prepared by placing the appropriate amount of the sodium salt of compound 1 in the capsule. The enteric-coating material was prepared by dissolving cellulose acetate phthalate in a mixture of isopropyl alcohol and dichloromethane. The entire capsule was dipped in the enteric-coating material, and the isopropyl alcohol and dichloromethane were allowed to evaporate. The dosage forms were administered to the animals and the concentration of the omeprazole in the blood was determined as described in the oral bioavailability determination of Example 2. The maximum concentration of omeprazole (Cₘₐₓ) and the total area under the curve (AUC) for the animals receiving both enterically coated and non-enterically coated oral dosage forms is presented in Table 4B. In both dogs and monkeys, both the Cmax and the AUC are higher for the non-enterically coated dosage form. While not intending to be bound in any way by theory, these results demonstrate that the prodrugs disclosed herein are stable enough that a sufficient quantity of the drug can be systemically delivered to the animal without enterically coating the prodrug, and that enteric coating may be omitted for the prodrugs if desired.

TABLE 4B
Effect of Enteric Coating on Systemic Omeprazole Concentration
Following Oral Administration of Compound 1 Capsules

| | Cₘₐₓ Omeprazole/Dose | | AUC Omeprazole/Dose | |
|---|---|---|---|---|
| | (ng/mL/mg/kg) | | (ng · hr/mL/mg/kg) | |
| | Enteric | Regular | Enteric | Regular |
| Animal | Coating | Capsule | Coating | Capsule |
| Dog | 22.5 ± 7.3 | 29.2 ± 11.8 | 82.2 ± 18.4 | 91.3 ± 32.9 |
| Monkey | 6.09 ± 1.04 | 14.0 ± 17.1 | 18.9 ± 7.9 | 19.7 ± 8.8 |

### EXAMPLE 5

A solid dosage form comprising 40 mg of compound 1, having 50% of the prodrug in the form of the sodium salt, is orally administered daily to a person suffering from heartburn. Relief of pain begins to occur within about 1 day, and continues as long as the person takes the dosage form.

1. An oral dosage form comprising a prodrug of a proton pump inhibitor, said prodrug having a membrane permeability and said proton pump inhibitor having a membrane permeability, wherein the membrane permeability of the proton pump inhibitor is more than twice the membrane permeability of the prodrug, and said dosage form has a pH from 3 to 9.

2. The dosage form of claim 1 wherein said dosage form has a pH from 5 to 8.

3. The dosage form of claim 1 wherein said dosage form has a pH from 6 to 8.

4. The dosage form of claim 1 wherein said prodrug is not enterically coated.

5. The dosage form of claim 1 wherein the membrane permeability of the proton pump inhibitor is more than 10 times the membrane permeability of the prodrug.

6. The dosage form of claim 1 wherein the membrane permeability of the proton pump inhibitor is more than 100 times the membrane permeability of the prodrug.

7. The dosage form of claim 1 wherein the membrane permeability of the proton pump inhibitor is more than 150 times the membrane permeability of the prodrug.

8. The dosage form of claim 2 wherein the membrane permeability of the proton pump inhibitor is more than 100 times the membrane permeability of the prodrug.

9. The dosage form of claim 2 wherein the membrane permeability of the proton pump inhibitor is more than 150 times the membrane permeability of the prodrug.

10. The dosage form of claim 3 wherein the membrane permeability of the proton pump inhibitor is more than 100 times the membrane permeability of the prodrug.

11. The dosage form of claim 3 wherein the membrane permeability of the proton pump inhibitor is more than 150 times the membrane permeability of the prodrug.

12. The dosage form of claim 1 wherein the membrane permeability of the prodrug is less than 1×10⁻⁶ cm/sec.

13. The dosage form of claim 1 wherein the membrane permeability of the prodrug is less than 5×10⁻⁷ cm/sec.

14. The dosage form of claim 1 wherein the membrane permeability of the prodrug is less than 1×10⁻⁷ cm/sec.

15. The dosage form of claim 1 wherein the membrane permeability of the prodrug is less than 5×10⁻⁸ cm/sec.

16. The dosage form of claim 1 wherein the prodrug comprises a carboxylic acid or a pharmaceutically acceptable salt thereof.

17. The dosage form of claim 1 wherein the prodrug comprises a sulfonyl moiety.

18. The dosage form of claim 1 wherein the prodrug comprises a phenylsulfonyl moiety.

19. The dosage form of claim 1 wherein the prodrug comprises a phenylsulfonyl moiety and a carboxylic acid or a pharmaceutically acceptable salt thereof.

20. The dosage form of claim 1 wherein the proton pump inhibitor is selected from the group consisting of lansoprazole, esomeprazole, omeprazole, pantoprazole, and rabeprazole.

21. The dosage form of claim 1 wherein the proton pump inhibitor is lansoprazole.

22. The dosage form of claim 1 wherein the proton pump inhibitor is omeprazole.

23. The dosage form of claim 1 wherein the proton pump inhibitor is pantoprazole.

24. The dosage form of claim 1 wherein the proton pump inhibitor is rabeprazole.

25. The dosage form of claim 1 which comprises a mixture of the prodrug and the proton pump inhibitor.

26. The dosage form of claim 25 wherein the membrane permeability of the proton pump inhibitor is more than twice the membrane permeability of the prodrug.

27. The dosage form of claim 25 wherein the membrane permeability of the proton pump inhibitor is more than 10 times the membrane permeability of the prodrug.

28. The dosage form of claim 25 wherein the membrane permeability of the proton pump inhibitor is more than 100 times the membrane permeability of the prodrug.

29. The dosage form of claim 25 wherein the membrane permeability of the proton pump inhibitor is more than 150 times the membrane permeability of the prodrug.

30. The dosage form of claim 1 which further comprises a second prodrug of said proton pump inhibitor.

31. The dosage form of claim 30, wherein the two prodrugs have a membrane permeability ratio which is from 2 to 10.

32. The dosage form of claim 30, wherein the two prodrugs have a membrane permeability ratio which is from 10 to 100.

33. The dosage form of claim 30, wherein the two prodrugs have a membrane permeability ratio which is from 100 to 500.

34. A method of treating a disease or adverse condition affecting the gastrointestinal tract in a person comprising administering orally to said person a prodrug of a proton pump inhibitor wherein said prodrug is a carboxylic acid which comprises a phenylsulfonyl moiety, wherein said carboxylic acid is in a dosage form wherein at least 1% of said carboxylic acid is in the form of a pharmaceutically acceptable salt.

35. The method of claim 34, wherein at least 50% of the carboxylic acid is in the form of the pharmaceutically acceptable salt.

36. The method of claim 34, wherein at least 90% of the carboxylic acid is in the form of a pharmaceutically acceptable salt.

37. The method of claim 34 wherein said prodrug is not enterically coated.

38. The method of claim 34 wherein the proton pump inhibitor is selected from the group consisting of lansoprazole, omeprazole, pantoprazole, and rabeprazole.

39. The method of claim 34 wherein the proton pump inhibitor is lansoprazole.

40. The method of claim 34 wherein the proton pump inhibitor is omeprazole.

41. The method of claim 34 wherein the prodrug has a structure comprising

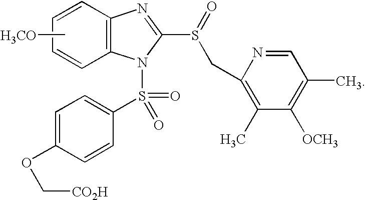

42. The method of claim 34 wherein the prodrug has a structure comprising

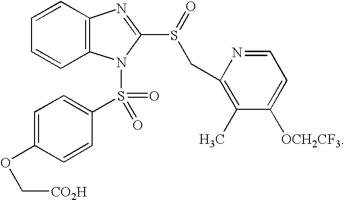

43. The method of claim 34 wherein the prodrug has a structure comprising

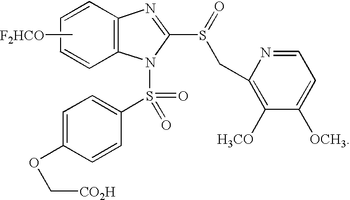

44. The method of claim 34 wherein the prodrug has a structure comprising

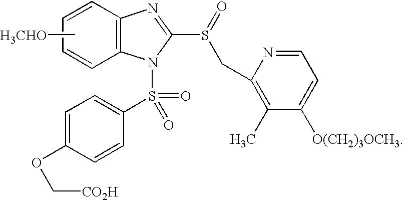

45. A method of inhibiting gastric acid secretion in a person comprising orally administering to said person a prodrug of a proton pump inhibitor, said prodrug having a membrane permeability which is less than 5×10⁻⁷ cm/sec.

46. The method of claim 45 wherein said prodrug comprises an acidic functional group having a pKₐ between 3 and 9 wherein at least 10% of said acidic functional group is in the form of a pharmaceutically acceptable salt.

47. The method of claim 46 wherein at least 50% of said acidic functional group is in the form of a pharmaceutically acceptable salt.

48. The method of claim 46 wherein at least 90% of said acidic functional group is in form of a pharmaceutically acceptable salt, and wherein at least 0.01% of the acidic functional group is in the acid form.

49. The method of claim 45 wherein said prodrug is not enterically coated in the dosage form in which it is administered.

50. The method of claim 45 wherein the membrane permeability of the prodrug is less than 1×10⁻⁷ cm/sec.

51. The method of claim 45 wherein the membrane permeability of the prodrug is less than 5×10⁻⁸ cm/sec.

52. The method of claim 45 wherein the prodrug comprises a carboxylic acid or a pharmaceutically acceptable salt thereof.

53. The method of claim 45 wherein the prodrug comprises a sulfonyl moiety.

54. The method of claim 45 wherein the prodrug comprises a phenylsulfonyl moiety and a carboxylic acid or a pharmaceutically acceptable salt thereof.

55. The method of claim 45 wherein the proton pump inhibitor is also administered to said person.

56. The method of claim 45 wherein a second prodrug is administered to said person.

57. The method of claim 56, wherein the two prodrugs have a membrane permeability ratio which is 2 or more.

58. The method of claim 56, wherein the two prodrugs have a membrane permeability ratio which is 10 or more.

59. The dosage form of claim 56, wherein the two prodrugs have a membrane permeability ratio which is 100 or more.

60. The method of claim 55 wherein the proton pump inhibitor has a membrane permeability which is more than twice the membrane permeability of the prodrug.

61. The method of claim 55 wherein the proton pump inhibitor has a membrane permeability which is more than 10 times the membrane permeability of the prodrug.

62. The method of claim 55 wherein the proton pump inhibitor has a membrane permeability which is more than 100 times the membrane permeability of the prodrug.

63. The method of claim 55 wherein the membrane permeability of the proton pump inhibitor is more than 150 times the membrane permeability of the prodrug.

64. A dosage form comprising a prodrug of a proton pump inhibitor wherein said prodrug comprises an acidic functional group and a sulfonyl moiety, wherein said dosage form is administered orally to a person, wherein at least 10% of said acidic functional group is in the form of a pharmaceutically acceptable salt.

65. The dosage form of claim 64 wherein at least 50% of said acidic functional group is in the form of a pharmaceutically acceptable salt.

66. The dosage form of claim 64 wherein at least 90% of said functional group is in the form of a pharmaceutically acceptable salt.

67. The dosage form of claim 64 wherein at least 90% of said functional group is in the form of a pharmaceutically acceptable salt and at least 0.01% of said functional group is in the acid form.

68. The dosage form of claim 64 which does not comprise any enteric coating.

69. The dosage form of claim 64 wherein the prodrug comprises a carboxylic acid or a pharmaceutically acceptable salt thereof.

70. The dosage form of claim 64 wherein the prodrug comprises a phenylsulfonyl moiety.

71. The dosage form of claim 64 wherein the prodrug comprises a phenylsulfonyl moiety and a carboxylic acid or a pharmaceutically acceptable salt thereof.

72. The dosage form of claim 64 wherein the proton pump inhibitor is selected from the group consisting of lansoprazole, omeprazole, pantoprazole, and rabeprazole.

73. The dosage form of claim 64 wherein the proton pump inhibitor is lansoprazole.

74. The dosage form of claim 64 wherein the proton pump inhibitor is omeprazole.

75. The dosage form of claim 64 comprising 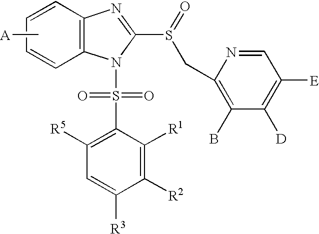
or a pharmaceutically acceptable salt thereof
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃.

76. The dosage form of claim 75 wherein R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

77. The dosage form of claim 64 wherein the prodrug has a structure comprising

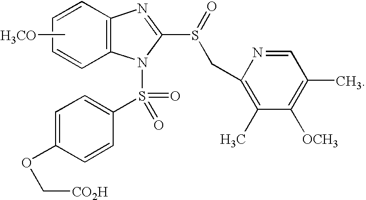

78. The dosage form of claim 64 wherein the prodrug has a structure comprising

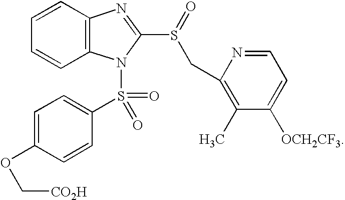

79. The dosage form of claim 64 wherein the prodrug has a structure comprising

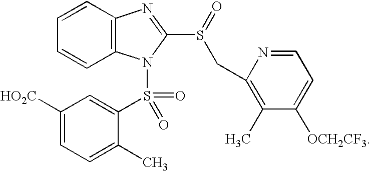

80. The dosage form of claim 64 wherein the prodrug has a structure comprising

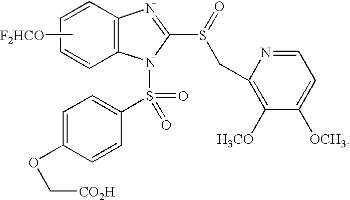

81. The dosage form of claim 64 wherein the prodrug has a structure comprising

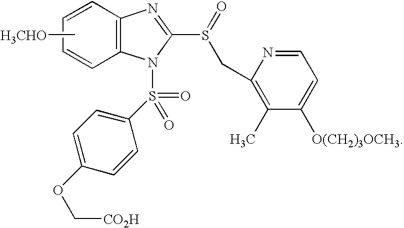

82. The dosage form of claim 67 wherein the prodrug has a structure comprising

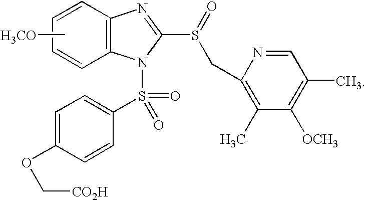

83. The dosage form of claim 67 wherein the prodrug has a structure comprising

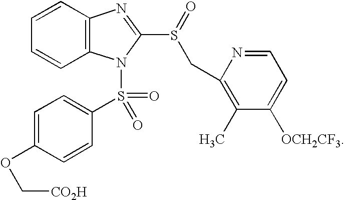

84. The dosage form of claim 67 wherein the prodrug has a structure comprising

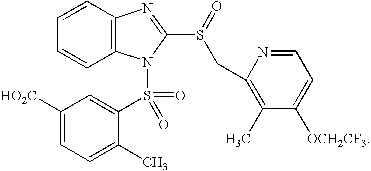

85. The dosage form of claim 67 wherein the prodrug has a structure comprising

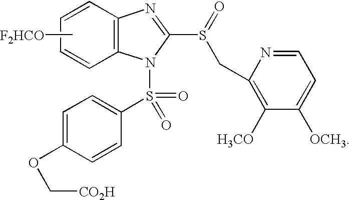

86. The dosage form of claim 67 wherein the prodrug has a structure comprising

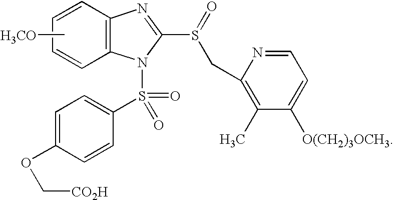

87. The dosage form of claim 68 wherein the prodrug has a structure comprising

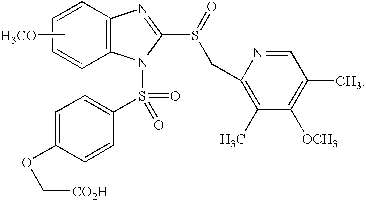

88. The dosage form of claim 68 wherein the prodrug has a structure comprising

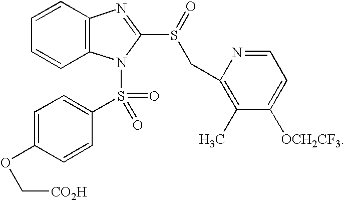

89. The dosage form of claim 68 wherein the prodrug has a structure comprising

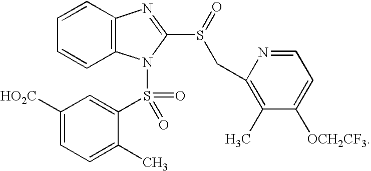

90. The dosage form of claim 68 wherein the prodrug has a structure comprising

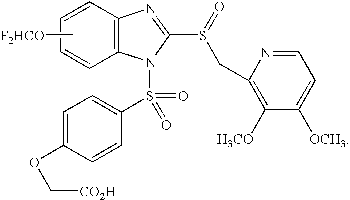

91. The dosage form of claim 68 wherein the prodrug has a structure comprising

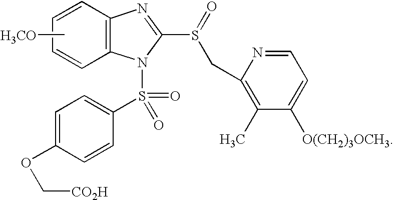

92. The dosage form of claim 25 wherein the ratio of the molar concentration of the prodrug to the molar concentration of the proton pump inhibitor is from 1 to 1000.

93. The dosage form of claim 30 wherein the ratio of the molar concentration of the two prodrugs is from 1 to 1000.

94. The dosage form of claim 34 wherein at least 10% of said acidic functional group is in the form of a pharmaceutically acceptable salt.
